(19) United States
(12) Patent Application Publication
(10) Pub. No.: US 2025/0069751 A1
(43) Pub. Date: Feb. 27, 2025

(54) METHOD AND SYSTEM FOR QUANTIFICATION OF SEVERITY OF A LUNG DISEASE

(71) Applicant: Veytel, Inc., Pittsburgh, PA (US)

(72) Inventors: Ellen K. HUGHES, Pittsburgh, PA (US); Catherine M. DIETZ, Redmond, WA (US); Kevin J. MITCHELL, Altoona, PA (US); Michael HOFFELDER, Norwich, NY (US)

(21) Appl. No.: 18/943,230

(22) Filed: Nov. 11, 2024

Related U.S. Application Data
(63) Continuation of application No. PCT/US2023/022106, filed on May 12, 2023.
(60) Provisional application No. 63/364,562, filed on May 12, 2022.

(51) Int. Cl. G16H 50/20; G16H 30/40; G16H 50/70

(52) U.S. Cl. CPC G16H 50/20; G16H 30/40; G16H 50/70

(57) ABSTRACT

A system, method, and computer program product for quantification of severity of a lung disease. An example aspect is configured to: provide an image file of a chest x-ray from a patient to a first artificial intelligence system to perform pre-processing of the image, wherein the lung is divided into at least four sections; provide each section to a second artificial intelligence system to generate a first density score and a first extent score of each section to calculate a first RALE score, wherein the second artificial intelligence system generates a density segmentation map of each section to calculate a second density score and a second extent score to calculate a second RALE score; analyze the first RALE score and the second RALE score; and display a result to a user.

### CROSS REFERENCE TO RELATED APPLICATIONS

This application is a bypass continuation application of International Application No. PCT/US2023/022106 filed on May 12, 2023, which claims priority to and the benefit of U.S. Provisional Patent Application No. 63/364,562 filed on May 12, 2022, entitled AUTOMATED QUANTIFICATION OF LUNG SEVERITY USING CHEST X-RAYS, which are expressly incorporated herein by reference in their entirety.

### FIELD OF THE INVENTION

This disclosure generally relates to methods and systems of quantifying severity of a lung disease in a medical image; and more particularly, methods and systems of quantifying severity of a lung disease in a chest x-ray.

### BACKGROUND

A chest x-ray is often used for assessing patients with acute respiratory illness, such as COVID-19, acute respiratory distress syndrome (ARDS), and pneumonia. While billions of chest x-rays may be performed annually, conventional chest x-ray analysis involves qualitative interpretation of features of a chest x-ray such as focal, patchy, or diffuse densities.

Conventional chest x-ray analysis requires manual examination of the x-ray by a physician or radiologist, wherein successful detection of abnormalities may depend on the radiologist's skill and/or experience. Thus, conventional analysis may lead to subjective, non-specific, and inconsistent diagnosis.

### BRIEF SUMMARY

The methods and systems of the present disclosure enable the quantification of severity of a lung disease. The methods and systems of the present disclosure may lead to objective and reproducible methods and systems to efficiently and accurately assess severity of a lung disease in a chest x-ray.

The present disclosure relates to a method for quantification of severity of a lung disease, the method comprising: providing an image file of a chest x-ray from a patient to a first artificial intelligence system to perform pre-processing of the image, wherein pre-processing comprises determining a vertex and performing segmentation of the image to define a lung boundary, and wherein the lung is divided into at least four sections; providing each section to a second artificial intelligence system to generate a first density score and a first extent score of each section, wherein the second artificial intelligence system is trained from a database comprising at least two reference image files of a chest x-ray, wherein the at least two reference image files comprise at least one annotation assigned by a physician, and wherein the second artificial intelligence system generates a density segmentation map of each section; calculating a first RALE score from the first density score and the first extent score of each section; analyzing the density segmentation map of each section to determine a second density score and a second extent score of each section to calculate a second RALE score of the image; analyzing the first RALE score and the second RALE score to determine a difference and an overall RALE score; and displaying a result to a user, wherein the result comprises the overall RALE score.

The presently disclosed systems and methods may be embodied as a system, method, or computer program product embodied in any tangible medium of expression having computer useable program code embodied in the medium.

### DESCRIPTION OF THE DRAWINGS

It is to be understood that both the foregoing summary and the following drawings and detailed description may be exemplary and may not be restrictive of the aspects of the present disclosure as claimed. Certain details may be set forth in order to provide a better understanding of various features, aspects, and advantages of the invention. However, one skilled in the art will understand that these features, aspects, and advantages may be practiced without these details. In other instances, well-known structures, methods, and/or processes associated with methods of practicing the various features, aspects, and advantages may not be shown or described in detail to avoid unnecessarily obscuring descriptions of other details of the invention.

The present disclosure may be better understood by reference to the accompanying drawing sheets, in which:

FIG. 1 is a flow chart of a method for quantification of severity of a lung disease, in accordance with certain aspects of the present disclosure.

FIG. 2 is a block diagram of the second artificial intelligence system, in accordance with certain aspects of the present disclosure.

FIG. 3A is a diagram of the first artificial intelligence system, in accordance with certain aspects of the present disclosure.

FIG. 3B is a diagram of the second artificial intelligence system, in accordance with certain aspects of the present disclosure.

FIG. 4 shows a screenshot of an annotator used by physicians to generate training data for the database, in accordance with certain aspects of the present disclosure.

FIG. 5A, 5B, 5C, 5D are density segmentation maps of each section of a lung, in accordance with certain aspects of the present disclosure.

FIG. 6 is a density segmentation map of the entire lung, in accordance with certain aspects of the present disclosure.

FIG. 7 is a block diagram of a system for quantification of severity of a lung disease, in accordance with certain aspects of the present disclosure.

FIG. 8 is a schematic diagram of a computer network system for quantification of severity of a lung disease according to the present disclosure.

### DETAILED DESCRIPTION

This disclosure generally describes methods and systems of quantification of severity of a lung disease. The methods and systems of the present disclosure may lead to objective and reproducible methods and systems to efficiently and accurately assess severity of a lung disease in a chest x-ray.

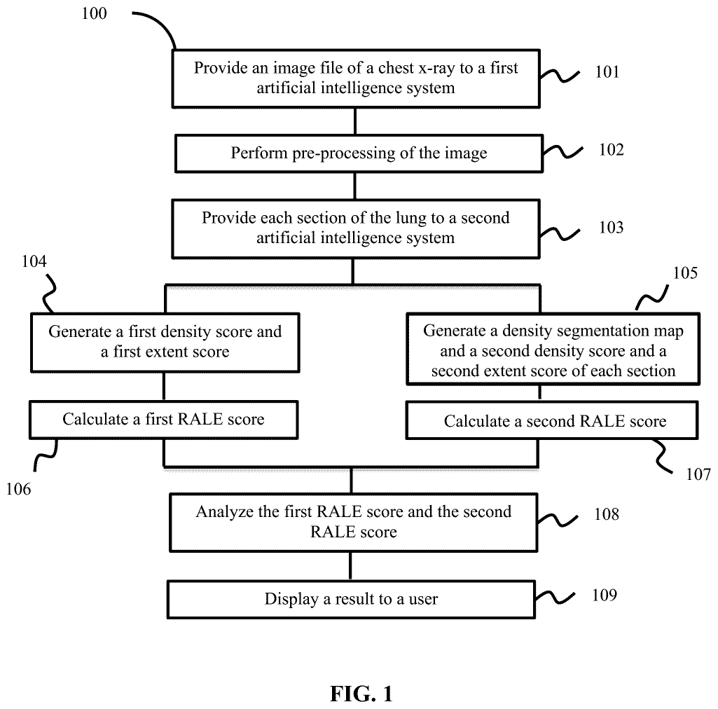

The present disclosure provides a method 100 (FIG. 1) for quantification of severity of a lung disease. The method 100 may provide an image file of a chest x-ray from a patient to a first artificial intelligence system 101. As used herein, an “artificial intelligence system” may include at least one computer vision algorithm and/or at least one deep learning network, wherein the at least one deep learning network may include at least one neural network having multiple layers. The use of “first” and “second” does not exclude one or more, two or more, or three or more artificial intelligence systems. The image file of a chest x-ray may include a data file, an image, and/or patient data, wherein the data and/or image may be formatted in any file format capable of storing a chest x-ray, including, but not limited to, DICOM, JPEG, TIGG, GIF, PNG, and the like.

The first artificial intelligence system may perform pre-processing of the image 102. Pre-processing may include lung segmentation, scale adjustment, rotation adjustment, angle adjustment, minimization of spurious equipment, such as IV lines, chemotherapy ports, EKG wiring, and the like, determining accurate vertex location, and the like. Segmentation may include defining a lung boundary wherein the lung is divided into at least four sections and anatomical features or image artifacts may be excluded except for the lung. While four segmented sections have been described, other segmentations with greater than four sections are possible and within the scope of the present disclosure. A vertex, as used herein, refers to the central point of the entire lung in a chest x-ray. Once a vertex is determined, the method 100 may divide the lung into at least four sections.

The first artificial intelligence system may be trained using pre-training data, wherein the pre-training data may comprise a collection of chest x-rays, such as MMIC-CXR. The first artificial intelligence system may train from a parameter used to characterize the image file of the present disclosure. A parameter may include, but is not limited to, a finding or lack of a finding, cancer and non-cancer, full breath taken by the patient or non-full breath taken by the patient, illness or healthy lung, and the like. A finding, as used herein, may refer to a visible region that is considered abnormal or unhealthy by a physician. The training of the first artificial intelligence system may train the method 100 to determine the weights and teach the appearance of a chest x-ray.

As generally understood in the art, weight is a parameter within a deep learning network that may transform input data within the network. A deep learning network may comprise a series of nodes. Each node may include a set of inputs, weight, and a bias value. As an input enters the node, it may be multiplied by a weight value, and the output may be observed or passed to the next node.

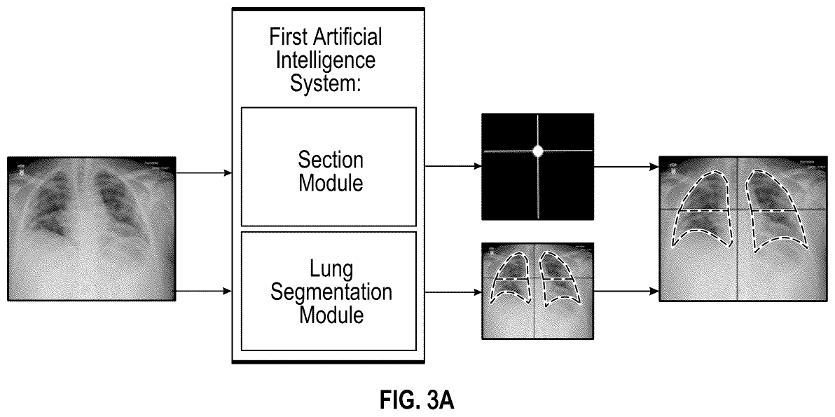

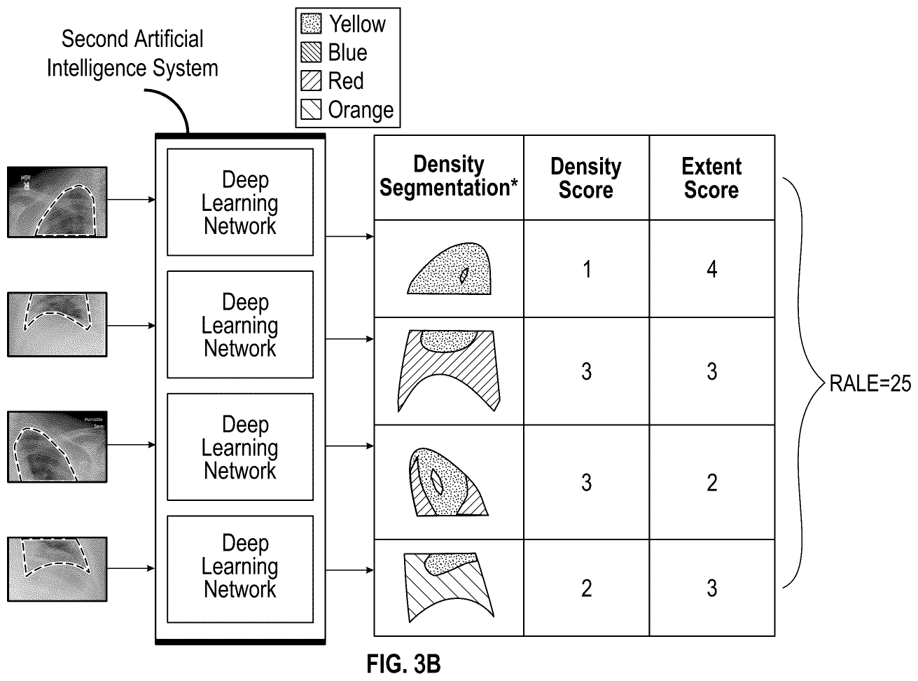

The first artificial intelligence system may include a section module having at least one computer vision algorithm and/or at least one deep learning network to determine the vertex and divide the lung into at least four sections. (FIG. 3A). The first artificial intelligence system may further include a lung segmentation module having at least one computer vision algorithm and/or at least one deep learning network to segment the lung (FIG. 3A). The section module and the lung segmentation module may work simultaneously to generate a segmented chest x-ray divided into four sections or quadrants (FIG. 3A).

Conventional methods and systems of quantification of severity of a lung disease require chest x-ray images taken in a specific pose, angle, or orientation. Thus, conventional methods may be unable to interpret a large subset of chest x-rays having different poses, angles, or orientation. Furthermore, a chest x-ray of a sick patient may exhibit variation when compared to a chest x-ray of a healthy patient. The methods and systems of the present disclosure may account for chest x-rays of different poses, angles, scales, orientation, and levels of patient health.

Conventional methods and systems may analyze the entire chest x-ray or segment the image into two sections. However, these methods and systems do not accurately assess chest x-rays wherein the lung is unbalanced in disease presentation. The methods and systems of the present disclosure may mitigate the risk posed by unbalanced disease presentation by segmenting and dividing the lung into four sections.

The method 100 may provide each section of the lung to a second artificial intelligence system 103. The second artificial intelligence system may generate a first density score and a first extent score of each section 104. As used herein, the term “extent” may be used interchangeable with “consolidation.” “Extent” may refer to the extent of alveolar opacities present in a chest x-ray or an amount of the highest opacity region. As used herein, “density” may refer to the density of alveolar opacities present in a chest x-ray. The second artificial intelligence system may then calculate a first RALE score from the first density score and the first extent score of each section 106.

As used herein, the term “RALE score”, may refer to a Radiographic Assessment of Lung Edema score used to evaluate the extent and density of alveolar opacities present in a chest x-ray. While a RALE score has been described, other non-invasive measurements to quantify severity of a lung disease are possible and within the scope of the present disclosure. A RALE score may range from 0 (low severity) to 48 (high severity).

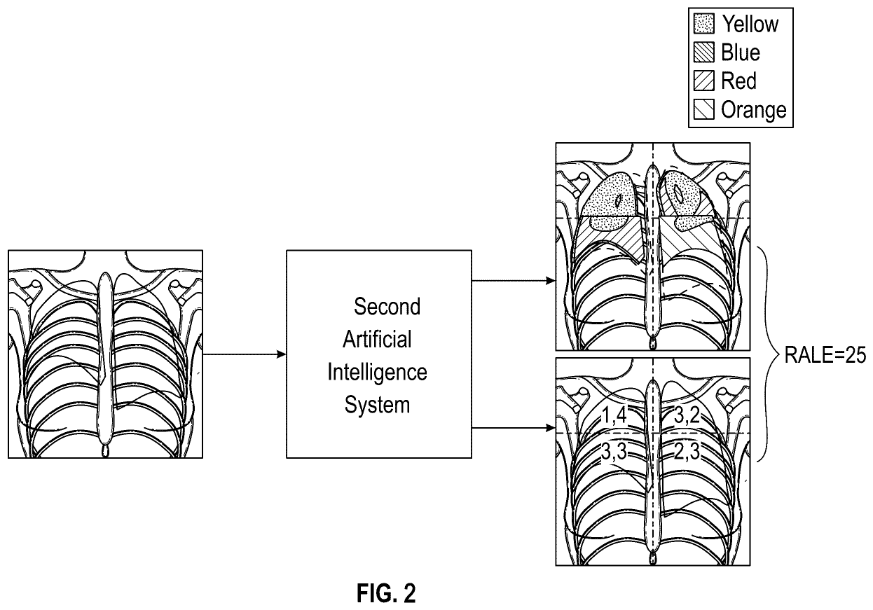

The second artificial intelligence system may include at least one deep learning network and/or at least one computer vision algorithm to determine the density and the extent of each section (FIG. 2). The second artificial intelligence system may include at least one individual deep learning network module having at least one deep learning network and/or at least one computer vision algorithm for each section of the lung (FIG. 3B) to determine the density and the extent of each section. The individual deep learning network module for each section may comprise RALE deep learning networks. Each RALE deep learning network module may be trained to directly compute the first density score and the first extent score of a section. Simultaneously, the at least one individual deep learning network module may generate a density segmentation map of each section 105 and determine the location of the densest point in each section to provide a high impact visualization for RALE deep learning explainability.

Each section may have a first extent score and a first density score. When the lung is segmented into four sections, the second artificial intelligence system may generate eight components of the first RALE score, including four first density scores and four first extent scores.

Conventional methods of determining a RALE score may report less than eight components of a RALE score, such as only reporting an overall RALE score for a chest x-ray, leading to inconsistent and inaccurate results. As such, conventional methods lack the ability to use RALE score components for training or for understanding how the RALE score was calculated. The methods and systems of the present disclosure may provide more consistent and more accurate results by dividing the RALE score into eight components and reporting the components to medical providers and databases for training artificial intelligence systems.

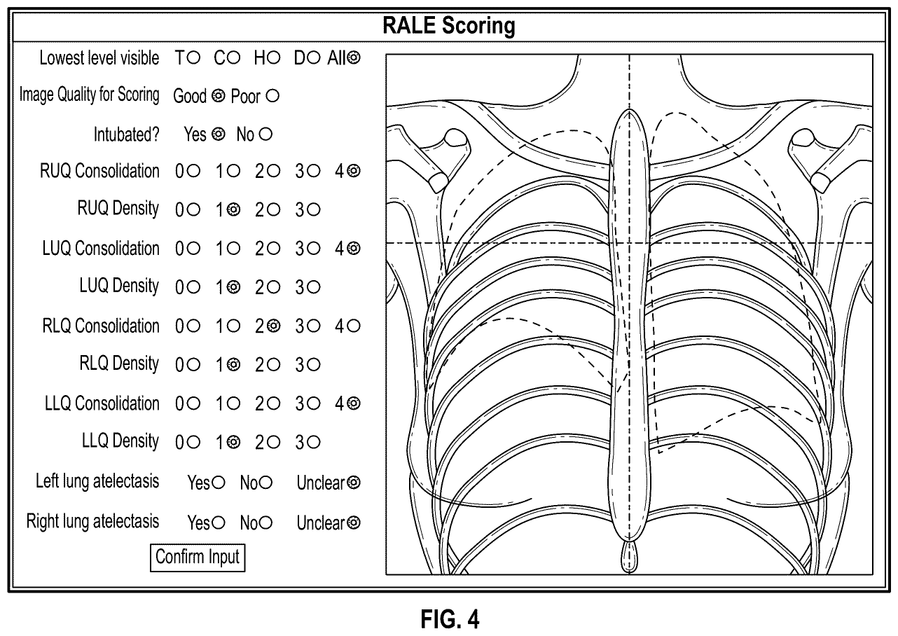

The second artificial intelligence system may be trained from a database including at least two reference image files of a chest x-ray. A reference image file may include a chest x-ray having at least one annotation assigned by a physician or radiologist using an annotator, wherein an annotator may include any system or computer program product wherein a physician or radiologist may assign a RALE score to a chest x-ray (FIG. 4). The database may comprise at least two reference image files of a chest x-ray, such as at least 2, 100, 1000, 2000, 4000, 6000, 8000, 10000, 50000, and at least 100000 reference image files of a chest x-ray. The reference image file may comprise a chest x-ray annotated by a physician or radiologist. The at least one annotation may include a first density score and a first extent score for each section or quadrant of the lung, presence of atelectasis, presence of intubation, image quality, and/or visibility level. The first density score and first extent score for each section of the reference image may be used to calculate an overall RALE score for the reference image. The second artificial intelligence system may be trained using the overall RALE score and/or the first density score and the first extent score of each section. The second artificial intelligence system may train from a combination of sections of the lung having at least one extent score and at least one density score. Accordingly, the second artificial intelligence system may train from at least one density score and at least one extent score annotated by a physician or radiologist. The annotated reference image files may comprise metadata to evaluate dataset statistics and avoid dataset build errors.

The second artificial intelligence system may be trained using a randomized set including at least 90% of the data within the database in order to randomize demographics and source of the image file.

In parallel to generating a first density score and a first extent score of each section 104 and calculating a first RALE score 106, the second artificial intelligence system may generate a density segmentation map of each section 105. The density segmentation map may comprise a visual or computer visual representation of the density in each section. The density segmentation map may comprise a 3-dimensional or 2-dimensional representation of the density of each section of the lung.

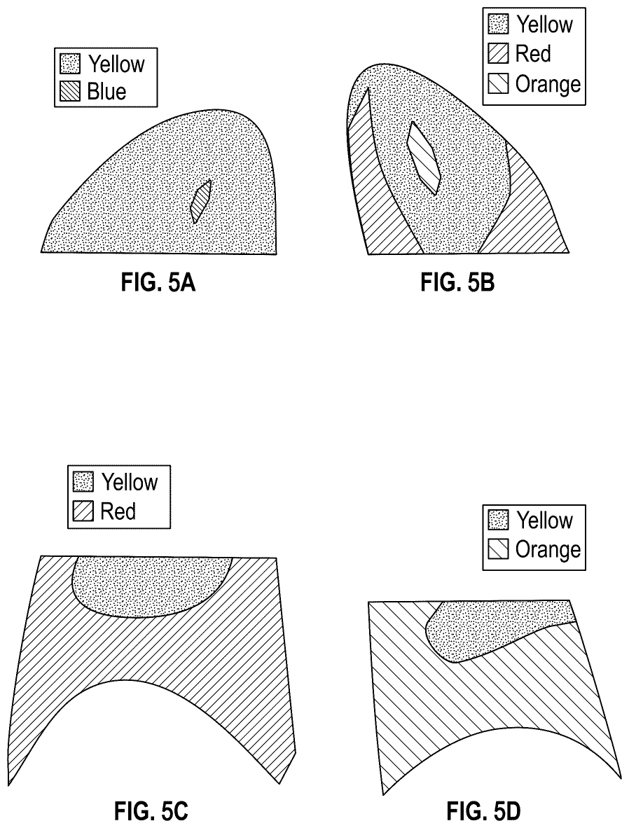

Each individual deep learning network module may detect the point of highest density in each quadrant and expand the highest density into a segmented region of the highest density. The density segmentation map may further comprise segmented regions of differing densities (FIGS. 5A-5D).

After generating a density segmentation map of each section 105, the method 100 may analyze the density segmentation map of each section to determine a second density score and a second extent score of each section. The second density score and second extent score of each section may be used to calculate a second RALE score of the image 107.

The method 100 may analyze the first RALE score and the second RALE score 108 to determine a difference and an overall RALE score. Determining a difference may include comparing the first RALE score and the second RALE score to determine the total amount of difference between the RALE scores. If the difference between the first RALE score and the second RALE score is 3 points, 2 points, 1 point, or 0 points, the first RALE score and the second RALE score may be characterized by the method 100 to be in agreement.

If the first RALE score and the second RALE score have a difference greater than 3, such as 4, 5, 6, 7, 8, 9, 10, 11, or greater than 12, the method 100 may recommend that the image file of the chest x-ray be reviewed and annotated by a physician or radiologist according to the present disclosure. Once the chest x-ray is reviewed and annotated by a physician or radiologist, the annotated chest x-ray image may be added to the database to further train the second artificial intelligence system.

The method 100 may determine an overall RALE score by calculating the minimum, maximum, or average of first RALE score and the second RALE score. While a minimum, maximum, or average is currently described, other calculations capable of determining an overall RALE score may be utilized by the methods and systems of the present disclosure.

When the difference between the first RALE score and the second RALE score is zero, one, two, or three, the method 100 may input the image file of the chest x-ray and the overall RALE score into the database, wherein the overall RALE score and/or any of the components of the first RALE score or the second RALE score may be used to train the second artificial intelligence system, creating positive feedback learning. Thus, the image file may become a reference image file. Accordingly, the method 100 may continuously train the second artificial intelligence system as the method analyzes an image file of a chest x-ray. As such, the methods and systems of the present disclosure may continuously learn while clinician involvement may only be needed when a difference greater than 3 points of the first RALE score and the second RALE score is detected. Accordingly, the methods and systems of the present disclosure may allow for exponentially larger training sets while minimizing clinician labor. Further, the methods and systems of the present disclosure may mitigate dataset biases currently limiting conventional use of deep learning in clinical practice, as the growing and diversified database may comprise patient chest x-rays with a wide spectrum of disease severity from multiple health centers in different geographic regions.

If the image file analyzed by the methods and systems of the present disclosure represents a new phenomenon, the first and second artificial intelligence systems may be retrained to account for the new phenomenon. A new phenomenon, as used herein, may include any new presentation of disease or illness.

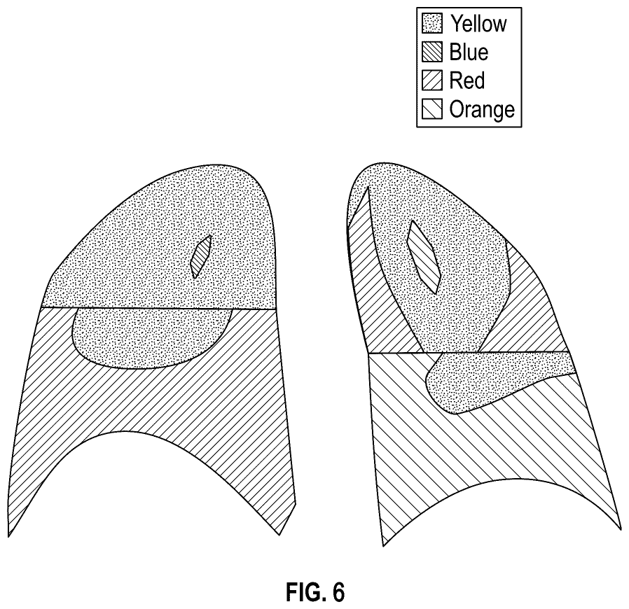

The method 100 may display a result to a user 109. The result may include the overall RALE score and/or the individual components of the first and second RALE scores. The result may also include displaying the density segmentation map of each section (FIGS. 5A-5D) on a graphical user interface. Each level of density may be represented by a different pattern or a different color, such as red, yellow, orange, blue, and the like. The density segmentation map of each section may be displayed as a 3-dimensional or 2-dimensional representation of the lung. The method may generate a density segmentation map as a 2-dimensional or 3-dimensional representation of the entire lung by combining each section (FIG. 6). Thus, the methods and systems of the present disclosure may enable explanatory deep learning, wherein the method 100 displays a high impact visualization of the density segmentation maps to physicians to aid in the understanding of the RALE calculations. The density segmentation maps may provide a visual of the highest density, providing a method less susceptible to misinterpretation, resulting in an increase in clinician trust.

The method 100 may result in a correlation to performance of trained clinicians or a RALE score confidence of at least 0.7, including, but not limited to, at least 0.8, 0.9, 0.91, 0.92, 0.93, 0.94, 0.95, 0.96, 0.97, 0.98, and at least 0.99.

The methods and systems of the present disclosure may be seamlessly incorporated into a clinical or hospital workplace. Conventional methods of determining a RALE score in a clinical or hospital workplace are laborious, subjective, inconsistent, non-specific, and rely on a radiologist's skill and/or experience. The systems and methods of the present disclosure address the problems present in the prior art by providing quantitative severity assessment of a lung disease with no additional work required on the part of a physician or radiologist. The methods and systems of the present disclosure may provide an objective, automated, real-time, accurate, consistent, workplace agnostic, and/or device agnostic RALE score.

Conventional methods of determining a RALE score may inaccurately define the vertex of a lung, especially when the chest x-ray may have a specific lung disease. An inaccurate vertex location may result in an incorrect RALE score. Accordingly, the first artificial intelligence system of the present disclosure provides automated and accurate determination of the vertex location.

The method may minimize training errors of the artificial intelligence systems of the present disclosure by pre-processing and calculating a first RALE score and a second RALE score.

The methods and systems of the present disclosure may be incorporated into the clinical workflow and may be integrated into conventional or future visualization software such as CERNER. The methods and systems of the present disclosure may be incorporated into chest x-ray acquisition systems to determine a RALE score in real-time once a chest x-ray is obtained.

Conventional methods and systems may include diagnosis of a specific lung disease or may simply aid in the diagnosis of a specific lung disease. The methods and systems of the present disclosure may accurately and consistently determine lung severity regardless of the lung disease present in a chest x-ray.

While conventional methods of determining a RALE score are labor intensive and inconsistent, the methods and systems of the present disclosure provide semi-supervised learning by coupling explanatory deep learning density segmentation with RALE score computation.

The methods and systems of the present disclosure may provide assessment of disease progression and measurement of treatment response over time. The methods and systems of the present disclosure may be used as a surrogate marker in clinical trials to enable faster and more efficient trials. As example, the methods and systems of the present disclosure may provide quantitative chest x-ray assessment and measurement of treatment effects directly on radiographic severity, such as a metric change from pre-treatment to post-treatment, in pharmaceutical and/or therapeutic trials. Thus, the methods and systems of the present disclosure provide surrogate metrics for treatment response, resulting in greater statistical power in testing treatment effects compared to conventional methods, which may result in reduced sample size requirements and more efficiently designed clinical trials, as reliable surrogate endpoints may result in significant cost and time savings for treatment testing.

The methods and systems of the present disclosure may be incorporated into integrated delivery networks.

The methods and systems of the present disclosure may be used in a triage situation to provide real-time quantification of lung severity prior to diagnosis of a specific lung disease.

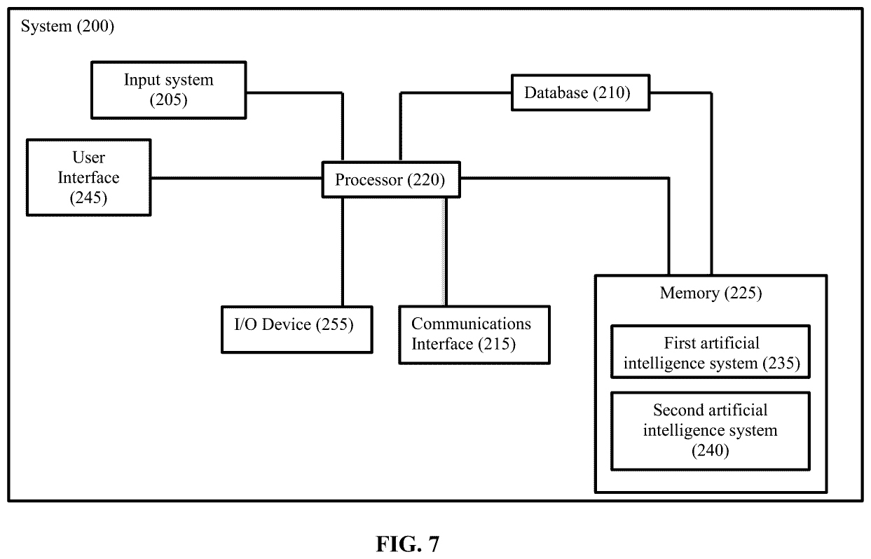

FIG. 7 is a block diagram that schematically illustrates a system 200 of the present disclosure for quantifying severity of a lung disease. The system 200 may include an input system 205. The input system 205 may comprise any system capable of allowing users to input and receive image files of a chest x-ray, including, but not limited to, cloud storage, Hospital PACS, computer, smartphone, web, any x-ray system, and the like. The image file of a chest x-ray may include a data file, an image, and/or patient data, wherein the data and/or image may be formatted in any file format capable of storing a chest x-ray, including, but not limited to, DICOM, JPEG, TIGG, GIF, PNG, and the like.

Systems of the present disclosure may be incorporated into the clinical workflow and may be integrated into conventional or future visualization software such as CERNER. The systems of the present disclosure may be incorporated into chest x-ray acquisition systems to determine an overall RALE score and/or RALE score components in real-time once a chest x-ray is obtained.

The system 200 may comprise a database 210. The database 210 may include at least two reference image files of a chest x-ray. A reference image file may comprise a chest x-ray having a an overall RALE score and/or RALE score components assigned by a physician or radiologist (FIG. 4). The database may comprise at least two reference image files of a chest x-ray, such as at least 2, 100, 1000, 2000, 4000, 6000, 8000, 10000, 50000, and at least 100000 reference image files of a chest x-ray. The reference image file may comprise a chest x-ray annotated by a physician or radiologist.

The system 200 may include one or more network and/or communications interface 215 for communicating with various computing entities, such as by communicating data, content, information, and/or similar terms used herein interchangeably that may be transmitted, received, operated on, processed, displayed, stored, and/or the like.

The system 200 may comprise a processor 220 that interfaces with memory 225 (which may be separate from or included as part of processor 220). The memory 225 may also employ cloud-based memory. In one aspect, the system may connect to a base station that includes memory and processing capabilities. The system may further comprise an I/O device 230. The processor 220 may interface with the database 210 according to the methods and systems of the present disclosure.

Memory 225 has stored therein a number of routines that are executable by processor 220. The processor 220, in communication with the memory 225, may be configured to execute a first artificial intelligence system 235. The first artificial intelligence system 235 may include program instructions or computer program code executable by processor 220 to perform pre-processing of an image file of a chest x-ray provided by the input system 205. Pre-processing may be performed according to the methods of the present disclosure.

The processor 220, in communication with the memory 225 may be configured to execute a second artificial intelligence system 240. The system 200 may include computer-executable instructions to provide each section of the lung to the second artificial intelligence system 240 to generate a first density score and a first extent score of each section according to the methods of the present disclosure. The second artificial intelligence system 240 may be trained from the database 210 according to the methods of the present disclosure.

The memory 225 may further include computer-executable instructions to calculate a first RALE score from the first density score and the first extent score of each section according to the methods of the present disclosure. The second artificial intelligence system 240 may include computer-executable instructions, executable by the processor 220 to generate and analyze a density segmentation map of each section to determine a second density score and a second extent score of each section to calculate a second RALE score of the image file according to the methods and systems of the present disclosure.

The processor 220, in communication with the memory 225, may be configured to execute program instructions to analyze the first RALE score and the second RALE score to determine a difference and an overall RALE score according to the methods of the present disclosure. The processor 220, in communication with the memory 225, may be configured to execute program instructions to display a result to a user on a graphical user interface 245 according to the methods of the present disclosure. The graphical user interface may include an output system, wherein the output system may comprise any system capable of receiving RALE score data, such as cloud storage, computer, medical device, smart phone, healthcare data systems, and/or the like, wherein RALE score data may comprise the overall RALE score, the first RALE score, the second RALE score, and/or at least one component of the first RALE score and the second RALE score.

Processor 220 may be one or more microprocessors, microcontroller, an application specific integrated circuit (ASIC), a circuit containing one or more processing components, a group of distributed processing components, circuitry for supporting a microprocessor, or other suitable processing device that interfaces with memory 225. Processor 220 is also configured to execute computer code stored in memory 225 to complete and facilitate the activities described herein.

The system 200 may include an I/O device 255, wherein an I/O device 255 (including, but not limited to, keyboards, displays, pointing devices, DASD, tape, CDs. DVDs, thumb drives and other memory media, etc.) may be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards may be just a few of the available types of network adapters.

As will be appreciated by one skilled in the art, the present disclosure may be embodied as a system, method, or computer program product. Accordingly, the present invention may take the form of an entirely hardware embodiment, an entirely software embodiment (including firmware, resident software, micro-code, etc.), or an embodiment combining software and hardware aspects that may all generally be referred to herein as a “system.” Furthermore, the present disclosure may take the form of a computer program product embodied in any tangible medium of expression having computer useable program code embodied in the medium. The computer program product of the present disclosure may comprise at least one non-transitory computer readable medium including program instructions that, when executed by at least one processor, cause the at least one processor to perform the methods of the present disclosure.

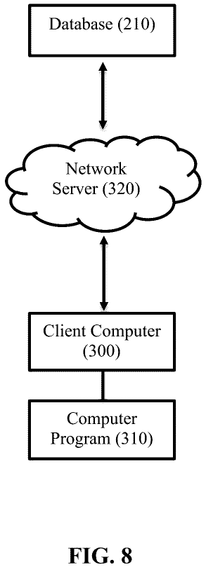

FIG. 8 depicts a schematic diagram of a computer network system of the present disclosure comprising a client computer 300 and a computer program 310 configured to execute the methods and systems of the present disclosure. The client computer 300 may be any device capable of executing the computer program 310 of the present disclosure. The system may interface with at least one network server 320, wherein the at least one network server 320 may interface with a database 210 and the system.

Although the depicted system is shown and described herein with certain components and functionality, other aspects of the system may be implemented with fewer or more components or with less or more functionality. Some aspects of the system may comprise a plurality of network servers 320, a plurality of networks, and a plurality of databases 210. Some aspects may include similar components arranged in another manner to provide similar functionality in one or more aspects.

The client computer 300 manages the interface between a system user and the computer program 310 and network server 320. Although the present disclosure is described with regard to a “computer”, it should be noted that optionally any device featuring a data processor and the ability to execute one or more instructions may be described as a computer, including but not limited to any type of personal computer (PC), a server, a distributed server, a virtual server, a cloud computing platform, a cellular telephone, an IP telephone, a smartphone, a mobile device, or a personal digital assistant (PDA). Any two or more of such devices in communication with each other may optionally comprise a network or a computer network.

The network may communicate traditional block I/O, for example, over a storage area network (SAN). The network may also communicate file I/O, for example, using a transmission control protocol/internet protocol (TCP/IP) network or similar communication protocol. In one aspect, the storage system includes two or more networks. In another aspect, the client computer 300 is connected directly to a network server 320 via a backplane or system bus. In one aspect, the network server 610 includes a cellular network, other similar types of networks, or combinations thereof.

The computing system may include clients and servers. A client and server are generally remote from each other and typically interact through a communication network. The relationship of client and server arises by virtue of computer programs running on the respective computers and having a client-server relationship to each other. In some aspects, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

Any combination of one or more computer useable or computer readable medium(s) may be utilized. The computer-useable or computer-readable medium may be, for example but not limited to, an electronic, magnetic, optical, electromagnetic, infrared, or semiconductor system, apparatus, device, or propagation medium. Computer-readable medium may also be an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CDROM), an optical storage device, a transmission media such as those supporting the Internet or an intranet, a magnetic storage device, a digital versatile disk (DVD), a memory stick, a floppy disk, a mechanically encoded device such as punch-cards or raised structures in a groove having instructions recorded thereon, and any suitable combination of the foregoing. Note that the computer-useable or computer-readable medium may be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-useable or computer-readable medium may be any medium that can contain, store, communicate, propagate, or transport the program for use by or in connection with the instruction execution system, apparatus, or device. The computer-useable program code may be transmitted using any appropriate medium, including but not limited to wireless, wireline, optical fiber cable, RF, etc.

Computer program code for carrying out operations of the presently disclosed invention may be written in any combination of one or more programming languages. The programming language may be, but is not limited to, object-oriented programming languages (Java, Smalltalk, C++, etc.) or conventional procedural programming languages (“C” programming language, etc.). The program code may execute entirely on a user's computer, partly on the user's computer, as a stand-alone software package, partly on a user's computer and partly on a remote computer, or entirely on the remote computer or server. In the latter scenario, the remote computer may be connected to the user's computer through any type of network, including a local area network (LAN) or a wide area network (WAN), or the connection may be made to an external computer, which may include through the Internet using an Internet Services Provider. In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

The systems and methods of the present disclosure may process data on any commercially available computer. In other aspects, a computer operating system may include, but is not limited to, Linux, Windows, UNIX, Android, MAC OS, and the like. In one aspect of the present disclosure, the forgoing processing devices or any other electronic, computation platform of a type designed for electronic processing of digital data as herein disclosed may be used.

Aspects of the present disclosure are described with reference to flowchart illustrations and/or block diagrams of methods, systems, and computer program products according to aspects of the present disclosure. It will be understood that each block of the flowchart illustrations and/or block diagrams, and combination of blocks in the flowchart illustrations and/or block diagrams, may be implemented by computer program instructions. These computer program instructions may be provided to a processor of a general-purpose computer, special purpose computer, or other programmable data processing apparatus to produce a machine, which the instructions execute via the processor of the computer or other programmable data processing apparatus allowing for the implementation of the steps specified in the flowchart and/or block diagram blocks or blocks.

Various embodiments of the present disclosure may be implemented in a data processing system suitable for storing and/or executing program code that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

Computer readable program instructions described herein may be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, among others.

### Definitions

Unless otherwise defined, all terms (including technical and scientific terms) used herein have the same meaning as commonly understood by one of ordinary skill in the art to which this disclosure belongs. As such, terms, such as those defined by commonly used dictionaries, should be interpreted as having a meaning that is consistent with their meaning in a context of a relevant art and should not be interpreted in an idealized or overly formal sense unless expressly so defined herein.

As used herein, the term “lung disease” may comprise any problem in the lungs that prevents the lungs from working properly, including, but not limited to COVID-19, acute respiratory distress syndrome (ARDS), pneumonia, respiratory syncytial virus (RSV), and the like.

As used herein, the term “user” refers to any person, entity, corporation, individual, institution, medical provider, medical facility, physician, physician assistant, nurse, nurse practitioner, doctor, patient, and the like capable of utilizing the methods and systems of the present disclosure.

As used herein, the term “patient” refers to any animal or human capable of receiving medical assistance or diagnostics according to the methods and systems of the present disclosure.

As used herein, the term “and/or” includes any and all combinations of one or more of the associated listed items. Likewise, as used in the following detailed description, the term “or” is intended to mean an inclusive “or” rather than an exclusive “or.” That is, unless specified otherwise, or clear from context, “X employs A or B” is intended to mean nay of the natural inclusive permutations. Thus, if X employs A; X employs B; or X employs both A and B, then “X employs A or B” is satisfied under any of the foregoing instances.

The terminology used herein is for the purpose of describing particular examples only and is not intended to be limiting. As used herein, the singular forms “a”, “an”, and “the” may be intended to include the plural forms as well, unless the context clearly dictates otherwise. As example, “a” image file” may comprise one or more image files, and the like.

The terms “comprises”, “comprising”, “including”, “having”, and “characterized by”, may be inclusive and therefore specify the presence of stated features, elements, compositions, steps, integers, operations, and/or components, but do not preclude the presence or addition of one or more other features, integers, steps, operations, elements, components, and/or groups thereof. Although these open-ended terms may be to be understood as a non-restrictive term used to describe and claim various aspects set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, described herein also specifically includes embodiments consisting of, or consisting essentially of, such recited compositions, materials, components, elements, features, integers, operations, and/or process steps. In the case of “consisting of”, the alternative embodiment excludes any additional compositions, materials, components, elements, features, integers, operations, and/or process steps, while in the case of “consisting essentially of”, any additional compositions, materials, components, elements, features, integers, operations, and/or process steps that materially affect the basic and novel characteristics may be excluded from such an embodiment, but any compositions, materials, components, elements, features, integers, operations, and/or process steps that do not materially affect the basic and novel characteristics may be included in the embodiment.

Any method steps, processes, and operations described herein may not be construed as necessarily requiring their performance in the particular order discussed or illustrated, unless specifically identified as an order of performance. It is also understood that additional or alternative steps may be employed, unless otherwise indicated.

In addition, features described with respect to certain example embodiments may be combined in or with various other example embodiments in any permutational or combinatory manner. Different aspects or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC. BC, or ABC, and if order is important in a particular context, also BA, CA, CB, CBA, BCA, ACB, BAC, or CAB. Continuing with this example, expressly included may be combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

Aspects of the present disclosure may be described herein with reference to flowchart illustrations and/or block diagrams of methods, apparatus (systems), and computer program products according to aspects of the disclosure. It will be understood that each block of the flowchart illustrations and/or block diagrams, and combinations of blocks in the flowchart illustrations and/or block diagrams, may be implemented by computer readable program instructions. The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The flowchart and block diagrams in the Figures illustrate the architecture, functionality, and operation of possible implementations of systems, methods, and computer program products according to various aspects of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, may be implemented by special purpose hardware-based systems that perform the specified functions or acts or carry out combinations of special purpose hardware and computer instructions.

Words such as “then,” “next,” etc. are not intended to limit the order of the steps; these words may be simply used to guide the reader through the description of the methods. Although process flow diagrams may describe the operations as a sequential process, many of the operations may be performed in parallel or concurrently. In addition, the order of the operations may be re-arranged. A process may correspond to a method, a function, a procedure, a subroutine, a subprogram, etc. When a process corresponds to a function, its termination may correspond to a return of the function to the calling function or the main function.

In the description, certain details are set forth in order to provide a better understanding of various embodiments of the systems and methods disclosed herein. However, one skilled in the art will understand that these embodiments may be practiced without these details and/or in the absence of any details not described herein. In other instances, well-known structures, methods, and/or techniques associated with methods of practicing the various embodiments may not be shown or described in detail to avoid unnecessarily obscuring descriptions of other details of the various embodiments.

While specific aspects of the disclosure have been provided hereinabove, the disclosure may, however, be embodied in many different forms and should not be construed as necessarily being limited to only the embodiments disclosed herein. Rather, these embodiments may be provided so that this disclosure is thorough and complete, and fully conveys various concepts of this disclosure to skilled artisans.

Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

All numerical quantities stated herein may be approximate, unless stated otherwise. Accordingly, the term “about” may be inferred when not expressly stated. The numerical quantities disclosed herein may be to be understood as not being strictly limited to the exact numerical values recited. Instead, unless stated otherwise, each numerical value stated herein is intended to mean both the recited value and a functionally equivalent range surrounding that value. At the very least, and not as an attempt to limit the application of the doctrine of equivalents to the scope of the claims, each numerical value should at least be construed in light of the number of reported significant digits and by applying ordinary rounding processes. Typical exemplary degrees of error may be within 20%, 10%, or 5% of a given value or range of values. Alternatively, the term “about” refers to values within an order of magnitude, potentially within 5-fold or 2-fold of a given value. Notwithstanding the approximations of numerical quantities stated herein, the numerical quantities described in specific examples of actual measured values may be reported as precisely as possible. Any numerical values, however, inherently contain certain errors necessarily resulting from the standard deviation found in their respective testing measurements.

All numerical ranges stated herein include all sub-ranges subsumed therein. For example, a range of “1 to 10” or “1-10” is intended to include all sub-ranges between and including the recited minimum value of 1 and the recited maximum value of 10 because the disclosed numerical ranges may be continuous and include every value between the minimum and maximum values. Any maximum numerical limitation recited herein is intended to include all lower numerical limitations. Any minimum numerical limitation recited herein is intended to include all higher numerical limitations.

Features or functionality described with respect to certain example embodiments may be combined and sub-combined in and/or with various other example embodiments. Also, different aspects and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, may be at least partially performed via at least one entity or actor in any manner.

All documents cited herein may be incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this application.

While particular embodiments have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications, and substitutions. This application including the appended claims is therefore intended to cover all such changes and modifications that may be within the scope of this application.

### Aspects

Aspect 1: A method for quantification of severity of a lung disease, the method comprising: providing an image file of a chest x-ray from a patient to a first artificial intelligence system to perform pre-processing of the image, wherein pre-processing comprises determining a vertex and performing segmentation of the image to define a lung boundary, and wherein the lung is divided into at least four sections; providing each section to a second artificial intelligence system to generate a first density score and a first extent score of each section, wherein the second artificial intelligence system is trained from a database comprising at least two reference image files of a chest x-ray, wherein the at least two reference image files comprise at least one annotation assigned by a physician, and wherein the second artificial intelligence system generates a density segmentation map of each section; calculating a first RALE score from the first density score and the first extent score of each section; analyzing the density segmentation map of each section to determine a second density score and a second extent score of each section to calculate a second RALE score of the image; analyzing the first RALE score and the second RALE score to determine a difference and an overall RALE score; and displaying a result to a user.

Aspect 2: The method of claim 1, wherein the first artificial intelligence system is trained using pre-training data.

Aspect 3: The method according to any of the foregoing aspects, wherein the pre-training data comprises at least two image files of a chest x-ray.

Aspect 4: The method according to any of the foregoing aspects, wherein pre-training data comprises a finding or a lack of finding.

Aspect 5: The method according to any of the foregoing aspects, wherein the first RALE score and the second RALE score are in agreement, and wherein the image file and the overall RALE score are added to the database and used to continuously train the second artificial intelligence system.

Aspect 6: The method according to any of the foregoing aspects, wherein the first RALE score and the second RALE score are in agreement, and wherein the image file and the first density score and the first extent score of at least section are added to the database and used to continuously train the second artificial intelligence system.

Aspect 7: The method according to any of the foregoing aspects, wherein the first RALE score and the second RALE score are in agreement, and wherein the image file and the second density score and the second extent score of at least one section are added to the database and used to continuously train the second artificial intelligence system.

Aspect 8: The method according to any of the foregoing aspects, wherein the database comprises at least 2000 image files of a chest x-ray, and wherein the at least one annotation is an overall RALE score assigned by a physician.

Aspect 9: The method according to any of the foregoing aspects, wherein the database comprises at least 2000 image files of a chest x-ray, and wherein the at least one annotation is at least one density score and at least one extent score assigned by a physician.

Aspect 10: The method according to any of the foregoing aspects, wherein displaying the result to a user comprises displaying the density segmentation map of each section on a graphical user interface.

Aspect 11: The method according to any of the foregoing aspects, wherein displaying the result to a user comprises displaying the overall RALE score to the user on a graphical user interface.

Aspect 12: The method according to any of the foregoing aspects, wherein displaying the result to a user comprises displaying at least one first extent score and at least one first density score of at least one section to the user on a graphical user interface.

Aspect 13: The method according to any of the foregoing aspects, wherein displaying the result to a user comprises displaying at least one second extent score and at least one second density score of at least one section to the user on a graphical user interface.

Aspect 14: The method according to any of the foregoing aspects, wherein displaying the result to a user comprises displaying the density segmentation map of each section as a 3-dimensional and/or 2-dimensional representation of the lung on a graphical user interface.

Aspect 15: The method according to any of the foregoing aspects, wherein the method has a RALE score confidence of at least 0.9.

Aspect 16: The method according to any of the foregoing aspects, wherein the first RALE score and the second RALE score do not agree, and wherein the image file is annotated by a physician to generate an annotated image file, wherein the annotated image file is added to the database to train the second artificial intelligence system.

Aspect 17: The method according to any of the foregoing aspects, wherein the first artificial intelligence system comprises at least one computer vision algorithm.

Aspect 18: The method according to any of the foregoing aspects, wherein the artificial intelligence system comprises at least one deep learning network.

Aspect 19: The method according to any of the foregoing aspects, wherein the first artificial intelligence system comprises at least one computer vision algorithm and at least one deep learning network.

Aspect 20: The method according to any of the foregoing aspects, wherein the first artificial intelligence system comprises a section module and a lung segmentation module.

Aspect 21: The method according to any of the foregoing aspects, wherein the second artificial intelligence system comprises at least one computer vision algorithm.

Aspect 22: The method according to any of the foregoing aspects, wherein the second artificial intelligence system comprises at least one deep learning network.

Aspect 23: The method according to any of the foregoing aspects, wherein the second artificial intelligence system comprises at least one computer vision algorithm and at least one deep learning network.

Aspect 24: The method according to any of the foregoing aspects, wherein the second artificial intelligence system comprises at least one deep learning network module.

Aspect 25: The method according to any of the foregoing aspects, wherein the at least one deep learning network module comprises at least one computer vision algorithm and/or at least one deep learning network.

Aspect 26: The method according to any of the foregoing aspects, wherein the second artificial intelligence system comprises at least one deep learning network module for each section.

Aspect 27: The method according to any of the foregoing aspects, wherein the deep learning network comprises at least one neural network.

Aspect 28: The method according to any of the foregoing aspects, wherein the at least one neural network comprises multiple layers.

Aspect 29: A system for quantification of severity of a lung disease, comprising: a database having at least two reference images of a chest x-ray, at least one processor, at least one communications interface, a user interface, and at least one memory including computer program code, the at least one memory and computer program code configured to store a first artificial intelligence system, a second artificial intelligence system, and computer-executable instructions, the memory further configured to execute, with the processor, the instructions, wherein the instructions include: providing an image file of a chest x-ray from a patient to a first artificial intelligence system to perform pre-processing of the image, wherein pre-processing comprises determining a vertex and performing segmentation of the image to define a lung boundary, and wherein the lung is divided into at least four sections; providing each section to a second artificial intelligence system to generate a first density score and a first extent score of each section, wherein the second artificial intelligence system is trained from the database having at least two reference images, wherein the at least two reference image files comprise at least one annotation assigned by a physician, and wherein the second artificial intelligence system generates a density segmentation map of each section; calculating a first RALE score from the first density score and the first extent score of each section; analyzing the density segmentation map of each section to determine a second density score and a second extent score of each section to calculate a second RALE score of the image file; analyzing the first RALE score and the second RALE score to determine a difference and an overall RALE score; and displaying a result to a user on the user interface.

Aspect 30: The system of claim 29, wherein the first artificial intelligence system is trained using pre-training data.

Aspect 31: The system according to any of the foregoing aspects, wherein the pre-training data comprises at least two image files of a chest x-ray.

Aspect 32: The system according to any of the foregoing aspects, wherein the image file and the overall RALE score are added to the at least two reference image files to continuously train the second artificial intelligence system.

Aspect 33: The system according to any of the foregoing aspects, wherein pre-training data comprises a finding or a lack of finding.

Aspect 34: The system according to any of the foregoing aspects, wherein the first RALE score and the second RALE score are in agreement, and wherein the image file and the overall RALE score are added to the database and used to continuously train the second artificial intelligence system.

Aspect 35: The system according to any of the foregoing aspects, wherein the first RALE score and the second RALE score are in agreement, and wherein the image file and the first density score and the first extent score of at least section are added to the database and used to continuously train the second artificial intelligence system.

Aspect 36: The system according to any of the foregoing aspects, wherein the first RALE score and the second RALE score are in agreement, and wherein the image file and the second density score and the second extent score of at least one section are added to the database and used to continuously train the second artificial intelligence system.

Aspect 37: The system according to any of the foregoing aspects, wherein the database comprises at least 2000 image files of a chest x-ray, and wherein the at least one annotation is an overall RALE score assigned by a physician.

Aspect 38: The system according to any of the foregoing aspects, wherein the database comprises at least 2000 image files of a chest x-ray, and wherein the at least one annotation is at least one density score and at least one extent score assigned by a physician.

Aspect 39: The system according to any of the foregoing aspects, wherein displaying the result to a user comprises displaying the density segmentation map of each section on the user interface.

Aspect 40: The system according to any of the foregoing aspects, wherein displaying the result to a user comprises displaying the overall RALE score to the user on the user interface.

Aspect 41: The system according to any of the foregoing aspects, wherein displaying the result to a user comprises displaying at least one first extent score and at least one first density score of at least one section to the user on the user interface.

Aspect 42: The system according to any of the foregoing aspects, wherein displaying the result to a user comprises displaying at least one second extent score and at least one second density score of at least one section to the user on the user interface.

Aspect 43: The system according to any of the foregoing aspects, wherein displaying the result to a user comprises displaying the density segmentation map of each section as a 3-dimensional and/or 2-dimensional representation of the lung on the user interface.

Aspect 44: The system according to any of the foregoing aspects, wherein the method has a RALE score confidence of at least 0.9.

Aspect 45: The system according to any of the foregoing aspects, wherein the first RALE score and the second RALE score do not agree, and wherein the image file is annotated by a physician to generate an annotated image file, wherein the annotated image file is added to the database to train the second artificial intelligence system.

Aspect 46: The system according to any of the foregoing aspects, wherein the first artificial intelligence system comprises at least one computer vision algorithm.

Aspect 47: The system according to any of the foregoing aspects, wherein the artificial intelligence system comprises at least one deep learning network.

Aspect 48: The system according to any of the foregoing aspects, wherein the first artificial intelligence system comprises at least one computer vision algorithm and at least one deep learning network.

Aspect 49: The system according to any of the foregoing aspects, wherein the first artificial intelligence system comprises a section module and a lung segmentation module.

Aspect 50: The system according to any of the foregoing aspects, wherein the second artificial intelligence system comprises at least one computer vision algorithm.

Aspect 51: The system according to any of the foregoing aspects, wherein the second artificial intelligence system comprises at least one deep learning network.

Aspect 52: The system according to any of the foregoing aspects, wherein the second artificial intelligence system comprises at least one computer vision algorithm and at least one deep learning network.

Aspect 53: The system according to any of the foregoing aspects, wherein the second artificial intelligence system comprises at least one deep learning network module.

Aspect 54: The system according to any of the foregoing aspects, wherein the at least one deep learning network module comprises at least one computer vision algorithm and/or at least one deep learning network.

Aspect 55: The system according to any of the foregoing aspects, wherein the second artificial intelligence system comprises at least one deep learning network module for each section.

Aspect 56: The system according to any of the foregoing aspects, wherein the deep learning network comprises at least one neural network.

Aspect 57: The system according to any of the foregoing aspects, wherein the at least one neural network comprises multiple layers.

Aspect 58: A computer program product for quantification of severity of a lung disease, comprising at least one non-transitory computer readable medium including program instruction that, when executed by at least one processor, cause the at least one processor to: provide an image file of a chest x-ray from a patient to a first artificial intelligence system to perform pre-processing of the image, wherein pre-processing comprises determining a vertex and performing segmentation of the image to define a lung boundary, and wherein the lung is divided into at least four sections; provide each section to a second artificial intelligence system to generate a first density score and a first extent score of each section, wherein the second artificial intelligence system is trained from a database comprising at least two reference image files of a chest x-ray, wherein the at least two reference image files comprise a at least one annotation assigned by a physician, and wherein the second artificial intelligence system generates a density segmentation map of each section; calculate a first RALE score from the first density score and the first extent score of each section; analyze the density segmentation map of each section to determine a second density score and a second extent score of each section to calculate a second RALE score of the image file; analyze the first RALE score and the second RALE score to determine a difference and an overall RALE score; and display a result to a user on the user interface, wherein the result comprises the overall RALE score.

Aspect 59: A computer program product according to any of the foregoing aspects.

Aspect 60: The method, system, and the computer program product according to any of the foregoing aspects, wherein the first RALE score comprises eight components.

Aspect 61: The method, system, and the computer program product according to any of the foregoing aspects, wherein the second RALE score comprises eight components.

### EXAMPLES

### Example 1

The systems and methods of the present disclosure analyzed chest x-ray images for 595 patients with COVID-19 upon ICU or hospital admission at a University of Pittsburgh Medical Center facility. Following training by a senior reviewer, eight physicians at different levels of training scored an independent set of chest x-rays, followed by feedback on scores distribution, and then independent rescoring by inter-reviewer correlations and intraclass correlation coefficients (ICC) in two-way random-effects models, and kappa statistic for categorical variables. We used average RALE scores from two reviewers with closes agreement to train the methods and systems of the present disclosure following pre-training with MMIC-CXR dataset using classes of “no finding” or “clinical pathology observation”.

We found inter-rater agreement for overall RALE scores (correlation R=0.71, p<0.0001; ICC =0.84 m 95% confidence interval [0.82-0.89]m p<0.0001), which improved to excellent agreement of the overall RALE score of the methods and systems of the present disclosure to the reviewers' overall RALE score (correlation R=0.88, p<0.0001; ICC=0.91 [0.88-0.94], p<0.0001), with <1% CXRs showing large RALE discrepancies (≥15 point difference). Reviewers had moderate agreement on image quality (kappa=0.6) and fair agreement on presence of atelectasis (kappa=0.21). Under-penetrated CXRs had higher median RALE scores compared to well-penetrated images (p<0.01), and presence of atelectasis was associated with higher mean right lower quadrant density scores. We then trained a deep learning network with the RALE score annotations and obtained a Spearman correlation R=0.87 (p=1.7×10−7) between predicted and physician-annotated RALE scores.

We demonstrated the systems and methods of the present disclosure may reliably, accurately, and rapidly predict RALE scores of a chest x-ray.

What is claimed is:

1. A method for quantification of severity of a lung disease, the method comprising:
providing an image file of a chest x-ray from a patient to a first artificial intelligence system to perform pre-processing of the image, wherein pre-processing comprises determining a vertex and performing segmentation of the image to define a lung boundary, and wherein the lung is divided into at least four sections;
providing each section to a second artificial intelligence system to generate a first density score and a first extent score of each section, wherein the second artificial intelligence system is trained from a database comprising at least two reference image files of a chest x-ray, wherein the at least two reference image files comprise at least one annotation assigned by a physician, and wherein the second artificial intelligence system generates a density segmentation map of each section;
calculating a first RALE score from the first density score and the first extent score of each section;
analyzing the density segmentation map of each section to determine a second density score and a second extent score of each section to calculate a second RALE score of the image;
analyzing the first RALE score and the second RALE score to determine a difference and an overall RALE score; and
displaying a result to a user.

2. The method of claim 1, wherein the first artificial intelligence system is trained using pre-training data.

3. The method of claim 2, wherein the pre-training data comprises at least two image files of a chest x-ray.

4. The method of claim 2, wherein pre-training data comprises a finding or a lack of finding.

5. The method of claim 1, wherein the first RALE score and the second RALE score are in agreement, and wherein the image file and the overall RALE score are added to the database and used to continuously train the second artificial intelligence system.

6. The method of claim 1, wherein the first RALE score and the second RALE score are in agreement, and wherein the image file and the first density score and the first extent score of at least section are added to the database and used to continuously train the second artificial intelligence system.

7. The method of claim 1, wherein the first RALE score and the second RALE score are in agreement, and wherein the image file and the second density score and the second extent score of at least one section are added to the database and used to continuously train the second artificial intelligence system.

8. The method of claim 1, wherein the database comprises at least 2000 image files of a chest x-ray, and wherein the at least one annotation is an overall RALE score assigned by a physician.

9. The method of claim 1, wherein the database comprises at least 2000 image files of a chest x-ray, and wherein the at least one annotation is at least one density score and at least one extent score assigned by a physician.

10. The method of claim 1, wherein displaying the result to a user comprises displaying the density segmentation map of each section on a graphical user interface.

11. The method of claim 1, wherein displaying the result to a user comprises displaying the overall RALE score to the user on a graphical user interface.

12. The method of claim 1, wherein displaying the result to a user comprises displaying at least one first extent score and at least one first density score of at least one section to the user on a graphical user interface.

13. The method of claim 1, wherein displaying the result to a user comprises displaying at least one second extent score and at least one second density score of at least one section to the user on a graphical user interface.

14. The method of claim 1, wherein the method has a RALE score confidence of at least 0.9.

15. The method of claim 1, wherein the first RALE score and the second RALE score do not agree, and wherein the image file is annotated by a physician to generate an annotated image file, wherein the annotated image file is added to the database to train the second artificial intelligence system.

16. A system for quantification of severity of a lung disease, comprising:
a database having at least two reference images of a chest x-ray, at least one processor, at least one communications interface, a user interface, and at least one memory including computer program code, the at least one memory and computer program code configured to store a first artificial intelligence system, a second artificial intelligence system, and computer-executable instructions, the memory further configured to execute, with the processor, the instructions, wherein the instructions include:
providing an image file of a chest x-ray from a patient to a first artificial intelligence system to perform pre-processing of the image, wherein pre-processing comprises determining a vertex and performing segmentation of the image to define a lung boundary, and wherein the lung is divided into at least four sections;
providing each section to a second artificial intelligence system to generate a first density score and a first extent score of each section, wherein the second artificial intelligence system is trained from the database having at least two reference images, wherein the at least two reference image files comprise at least one annotation assigned by a physician, and wherein the second artificial intelligence system generates a density segmentation map of each section;
calculating a first RALE score from the first density score and the first extent score of each section;
analyzing the density segmentation map of each section to determine a second density score and a second extent score of each section to calculate a second RALE score of the image file;
analyzing the first RALE score and the second RALE score to determine a difference and an overall RALE score; and
displaying a result to a user on the user interface.

17. The system of claim 16, wherein the first artificial intelligence system is trained using pre-training data.

18. The system of claim 17, wherein the pre-training data comprises at least two image files of a chest x-ray.

19. The system of claim 17, wherein pre-training data comprises a finding or a lack of finding.

20. The system of claim 16, wherein the first RALE score and the second RALE score are in agreement, and wherein the image file and the overall RALE score are added to the database and used to continuously train the second artificial intelligence system.

21. The system of claim 16, wherein the first RALE score and the second RALE score are in agreement, and wherein the image file and the first density score and the first extent score of at least section are added to the database and used to continuously train the second artificial intelligence system.

22. The system of claim 16, wherein the first RALE score and the second RALE score are in agreement, and wherein the image file and the second density score and the second extent score of at least one section are added to the database and used to continuously train the second artificial intelligence system.

23. The system of claim 16, wherein the database comprises at least 2000 image files of a chest x-ray, and wherein the at least one annotation is an overall RALE score assigned by a physician.

24. The system of claim 16, wherein the database comprises at least 2000 image files of a chest x-ray, and wherein the at least one annotation is at least one density score and at least one extent score assigned by a physician.

25. The system of claim 16, wherein displaying the result to a user comprises displaying the density segmentation map of each section on the user interface.

26. The system of claim 16, wherein displaying the result to a user comprises displaying the overall RALE score to the user on the user interface.

27. A computer program product for quantification of severity of a lung disease, comprising at least one non-transitory computer readable medium including program instruction that, when executed by at least one processor, cause the at least one processor to:
provide an image file of a chest x-ray from a patient to a first artificial intelligence system to perform pre-processing of the image, wherein pre-processing comprises determining a vertex and performing segmentation of the image to define a lung boundary, and wherein the lung is divided into at least four sections;
provide each section to a second artificial intelligence system to generate a first density score and a first extent score of each section, wherein the second artificial intelligence system is trained from a database comprising at least two reference image files of a chest x-ray, wherein the at least two reference image files comprise at least one annotation assigned by a physician, and wherein the second artificial intelligence system generates a density segmentation map of each section;
calculate a first RALE score from the first density score and the first extent score of each section;
analyze the density segmentation map of each section to determine a second density score and a second extent score of each section to calculate a second RALE score of the image file;
analyze the first RALE score and the second RALE score to determine a difference and an overall RALE score; and
display a result to a user on the user interface.
